(19) United States
(12) Patent Application Publication
(10) Pub. No.: US 2026/0060914 A1
(43) Pub. Date: Mar. 5, 2026

(54) CONTOUR INJECTION COMPOSITION

(71) Applicant: Jong Cheol HONG, Seoul (KR)

(72) Inventor: Jong Cheol HONG, Seoul (KR)

(21) Appl. No.: 18/880,553

(22) PCT Filed: Jan. 9, 2023
(86) PCT No.: PCT/KR2023/000400

§ 371 Date: Nov. 2, 2025

(30) Foreign Application Priority Data
Sep. 1, 2022 (KR) 10-2022-0111036

(51) Int. Cl. A61K 8/66; A61K 8/19; A61K 8/41; A61K 8/44; A61K 8/49; A61K 8/55; A61K 8/9717; A61Q 19/06

(52) U.S. Cl. CPC A61K 8/66; A61K 8/19; A61K 8/41; A61K 8/44; A61K 8/4953; A61K 8/55; A61K 8/9717; A61Q 19/06; C12Y 302/01035

(57) ABSTRACT

The present invention relates to a contour injection composition, and more specifically, is characterized by an injection composition comprising a solvent, aminophylline, procaine, epinephrine, and hyaluronidase, the injection composition comprising, on the basis of the entire composition, 500 to 700 IU units hyaluronidase, and 0.05 to 0.15% (w/v) aminophylline, 0.01 to 0.03% (w/v) procaine, 0.001 to 0.006% (w/v) epinephrine, and the remainder the solvent, wherein the solvent is half saline and distilled water. According to the present invention, the injection composition has excellent fat decomposition and fat destruction effects and cellulite reduction effect, as well as effect in improving a circulation function, thereby reducing subcutaneous fat in a desired area, and having the advantages not only of improving body and facial lines by improving elasticity, but also not causing side effects such as redness, itchiness, swelling, skin depression, and skin sagging.

### TECHNICAL FIELD

The present invention relates to a contour injection composition, and more specifically, to a contour injection composition having excellent fat decomposition and fat destruction effects and excellent elasticity improvement effect without a side effect.

### TECHNICAL BACKGROUND

Modern people live in an era of energy overload, where the supply of energy exceeds the demand, as the mount of physical activities has been reduced due to the longer working hours and developed transportation caused by industrialization, while their eating habits have become westernized so that their food has been changed to high-calorie fast food represented by pizza, hamburgers, and cola.

Adipocytes in the human body are involved in the accumulation and release of energy: when there is an energy overload, the energy is stored as neutral fat within the adipocytes, and when there is an energy shortage, neutral fat is decomposed into glycerol and free fatty acids to be used as energy. Therefore, an energy overload leads to the accumulation of fat, which can cause health problems, such as lifestyle diseases caused by obesity, or cause personal physical complexes due to the localized accumulation of fat.

Representative methods for removing fat accumulated in the body include exercise therapy and diet therapy, but the above-described therapies are difficult to sustain, and when fat is accumulated in local regions such as the face, abdomen, and lower body, satisfactory results may not be obtained with the above-described therapies alone. As a result, various liposuction or lipolysis methods that can remove fat in local regions are continuously being developed.

Lipolytic injections, also known as celebrity injections or contour injections, refer to subcutaneous injections of substances that have lipolytic properties. The lipolytic injections are gaining popularity because they are effective for easy weight loss, body management, and decomposition of localized fat compared to exercise therapy or diet therapy.

The ingredients of conventional lipolytic injections included phosphatidylcholine, deoxycholic acid, and hyaluronidase.

However, the ingredients may have side effects such as swelling, hematoma, pain at the treatment site, itchiness and burning sensation at the injection site after treatment, and cell necrosis, and in the case of hyaluronidase, there is a problem that skin sagging or wrinkles may occur when high doses are used. Furthermore, it was difficult to obtain a satisfactory level of lipolytic effect with these injections alone.

Therefore, there is a need to develop a fat removal injection capable of effectively removing fat while minimizing the side effects of the ingredients used for lipolysis.

### RELATED ART DOCUMENTS

### Patent Document

(Patent document 0001) KR 10-2172439 B1
(Patent document 0002) KR 10-1706549 B1

### Problems to be Solved

Therefore, one object of the present invention is to provide a contour injection composition having excellent fat decomposition and fat destruction effects without a side effect in order to solve the various problems of the above-described conventional non-surgical method

Another object of the present invention is to provide a contour injection composition that improves body and facial lines by improving circulation function, reducing subcutaneous fat in a desired region, and improving elasticity.

### SUMMARY OF INVENTION

A contour injection composition according to the present invention is an injection composition including: a solvent, aminophylline, procaine, epinephrine, and hyaluronidase,
wherein, based on the entire composition,
the hyaluronidase is included in 500 to 700 IU units,
the aminophylline is included in an amount of 0.05% to 0.15% (w/v), the procaine is included in an amount of 0.01% to 0.03% (w/v), the epinephrine is included in an amount of 0.001% to 0.006% (w/v), and the solvent is included as the remainder,
wherein the solvent is half saline and distilled water.

Based on the entire composition, the composition further includes 2% to 4% (w/v) of choline alfoscerate.

Based on the entire composition, the composition further includes 3% to 6% (v/v) of carbonated water.

Based on the entire composition, the composition further includes 1% to 5% (v/v) of vitamin B1.

The half saline and distilled water are included at a volume ratio of 3:7.

The composition further includes 0.001% to 0.01% (w/v) of Asparagopsis taxiformis extract.

### Advantages of Invention

According to the contour injection composition of the present invention, there are advantages of exhibiting excellent fat decomposition and fat destruction effects and cellulite reduction effect as well as an effect in improving a circulation function, thereby reducing subcutaneous fat in a desired region, and improving body and facial lines by improving elasticity, without side effects such as redness, itchiness, swelling, skin depression, and skin sagging.

### DETAILED DESCRIPTION

Hereinafter, the present invention will be described in detail.

The present invention is an injection composition including a solvent, aminophylline, procaine, epinephrine, and hyaluronidase, and is characterized in that it effectively reduces subcutaneous fat and improves elasticity in a desired region through fat decomposition and fat destruction, cellulite reduction, and improvement of circulation function without side effects.

More specifically, the contour injection composition of the present invention is an injection composition including a solvent, aminophylline, procaine, epinephrine, and hyaluronidase, wherein, based on the entire composition, the hyaluronidase is contained in units of 500 to 700 IU, the aminophylline is included in an amount of 0.05% to 0.15% (w/v), the procaine is included in an amount of 0.01% to 0.03% (w/v), the epinephrine included in an amount of 0.001% to 0.006% (w/v), and the solvent is included as the remainder, and the solvent is half saline and distilled water.

It is to be noted that the “%” of the active ingredients in the present invention is not based on the entire injection solution of the ingredients that are commercially obtainable, but on the ingredients themselves. For example, the mixing ratio of the aminophylline refers to, not the mixing ratio based on the entire commercially obtainable aminophylline injection, but the mixing ratio of only aminophylline, which is the active ingredient in the aminophylline injection.

First, the hyaluronidase is a protein enzyme that hydrolyzes hyaluronic acid, which is the intercellular base material of connective tissues, and controls the binding force of the intercellular space to facilitate drug absorption and diffusion, effectively controls fibrosis related to cellulite, and decomposes adipocytes to correct body shape. However, the hyaluronidase may cause side effects such as hypersensitivity, dizziness, pain, swelling, itchiness, and erythema, and in case of high-dose administration, severe side effects such as skin sinking, skin sagging, and wrinkles may occur.

In the present invention, the hyaluronidase is preferably included in units of 500 to 700 IU. When it is included in less than 500 IU, the effect of decomposing fat tissue is minimal, and when it is included in more than 700 IU, as a high-concentration injection is administered to one region of the skin, the skin may become bumpy and skin sagging may occur.

The aminophylline is used to help decompose fat. The aminophylline may promote decomposition of fat through the following pathways. First, it may inhibit phosphodiesterase type IIIB to increase intracellular cyclic adenosine monophosphate (AMP) and induce protein kinase activation. Then, it may promote decomposition of fat by inhibiting adenosine receptors and may promote decomposition of fat by serving as a sympathomimetic agent (beta2-adrenergic agonist).

The aminophylline is preferably included in an amount of 0.05% to 0.15% (w/v) based on the entire composition. When the mixing ratio is less than 0.05% (w/v), decomposition of fat may not be promoted, and when it exceeds 0.15% (w/v), side effects such as skin sinking, edema, pain, and itchiness may occur due to excessive administration.

The procaine is used as a local anesthetic to alleviate itchiness and pain caused by skin inflammation.

The procaine is preferably included in an amount of 0.01% to 0.03% (w/v) based on the total composition. When the procaine is less than 0.01% (w/v), the anesthetic effect may be reduced, and when it exceeds 0.03% (w/v), the anesthetic time may be prolonged, which is not suitable for the purpose of the present invention, which is to enable a quick and easy procedure to quickly return to daily living, and side effects due to toxicity may occur.

The epinephrine has the effects of reducing local pain and constricting blood vessels, thereby preventing blood vessel damage and preventing severe bruising caused by injection.

The epinephrine is preferably included in an amount of 0.001% to 0.006% (w/v) based on the total amount of the composition, because when the amount is too little, a sufficient vasoconstriction effect may not be exhibited, and when it is excessive, side effects may rather be increased.

In the present invention, half saline and distilled water are used as the solvent.

The half saline is a physiological saline solution having a sodium chloride concentration of 0.4% by weight, and unlike general physiological saline having a sodium chloride concentration of 0.9% by weight, the half saline is a hypotonic solution.

In other words, when free water is used as a solvent in a pharmaceutical composition for fat decomposition injection, the osmolarity of the composition is too low, which may cause side effects, and when normal saline (0.9% NaCl) is used as a solvent, the osmolarity of the composition is too high, which causes pain, and it is difficult to exhibit the fat decomposition effect of a hypotonic solution.

Therefore, the present invention uses half saline and distilled water mixed at a volume ratio of 3:7 as a solvent, thereby decreasing side effects and reducing the occurrence of pain while providing an excellent fat decomposition effect.

The solvent is included as the remainder of the injection composition.

In addition, the composition of the present invention may further include pharmaceutically acceptable additives. The pharmaceutically acceptable additives of the present invention may include antiseptics, stabilizers, and preservatives, but are not limited thereto. It is obvious that the content and type of the additives may be appropriately selected by one of ordinary skill in the art within a known range within a scope that does not affect the stability or effectiveness of the active ingredients.

The term “local” as used herein refers to a part of the whole, and means a part of the human body. For example, it may include a subcutaneous region, and more specifically, it may include under the subocular regions, chin, cheekbones, deep cheekbones, upper abdomen, lower abdomen, the love handles, thighs, calves, the Chanel line, accessary breasts, arm fat, calves, ankles, and the like, but is not limited thereto.

The composition of the present invention may be administered by subcutaneous, intraperitoneal, and intramuscular injection.

In addition, the dosage of the composition of the present invention may vary depending on the conditions and weight of the patient, the type and severity of the disease, and the administration route and period. In addition, the composition of the present invention may be administered once a day or divided into several doses.

In addition, the composition of the present invention may be administered by injecting the injection at 4 to 12 points, and the injection point interval may be 3 to 10 cm, but is not limited thereto. At this time, the point refers to one point at which the invention is injected through a medical device such as a syringe for fat removal, and the point interval refers to the spatial gap between one point at which the injection is injected for fat removal and another point closest to thereto at which the injection is injected.

Since the composition of the present invention has sufficient dispersibility in the body when administered, even when it is not administered at a high density with a narrow injection point interval, even fat decomposition may be achieved, thereby minimizing side effects such as skin sagging.

Meanwhile, the contour injection composition of the present invention preferably further includes 2% to 4% (w/v) of choline alfoscerate based on the total amount of the composition.

The choline alfoscerate is a substance originally referred to as glycerylphosphorylcholine (GPC), which is a major component of neural membrane phospholipids and is also a choline precursor. In other words, it facilitates the supply of choline to synthesize acetylcholine, thereby improving neurotransmission function and restoring normal nerve function, and at the same time, the supply of glycerol-3-phosphate acts on the neural membrane phospholipids to restore the neural membrane and improve nerve function, and it also activates fat metabolism to promote fat decomposition. In addition, the choline alfoscerate prevents the decrease of skin elasticity.

In addition, the contour injection composition of the present invention preferably further contains 3% to 5% (v/v) of carbonated water based on the total amount of the composition.

The carbonated water, manufactured by injecting carbon dioxide into distilled water, helps destroy fat cells through carbon dioxide and improves circulation function. In addition, it prevents the decrease of skin elasticity

The contour injection composition of the present invention preferably further includes 3% to 5% (v/v) of vitamin B1 based on the total amount of the composition.

The vitamin B1 promotes circulation effect and helps in fat decomposition, and as an example, Beecom Hexa Injection may be used.

The contour injection composition of the present invention preferably further includes 0.001% to 0.01% (w/v) of Asparagopsis taxiformis extract based on the total amount of the composition.

The Asparagopsis taxiformis is a species belonging to red algae and is distributed worldwide in tropical and temperate seas. An extract of Asparagopsis taxiformis has the effect of promoting the decomposition of fat while reducing side effects and significantly improving elasticity through interaction with the above-described main active ingredients.

At this time, the Asparagopsis taxiformis extract is sufficiently extracted using water, ethanol, or a mixed solution thereof as a solvent, and since the method of preparing an extract from such a natural product is sufficiently known in the field to which this technology pertains, a detailed description thereof will be omitted.

The above-described injection composition of the present invention has the advantage of having excellent fat decomposition and fat destruction effects, cellulite reduction effects, and improving circulation function and elasticity, thereby reducing subcutaneous fat in a desired region and improving body and facial lines without side effects.

Hereinafter, the present invention will be described in detail through specific examples.

### Example 1

An injection composition was prepared by mixing 0.1% (w/v) aminophylline, 0.02% (w/v) procaine, 0.004% (w/v) epinephrine, 600 IU hyaluronidase, and a solvent as a remainder based on the total amount of the composition.

At this time, a mixture of half saline and distilled water at a volume ratio of 3:7 was used as the solvent.

### Example 2

An injection composition was prepared by performing the same procedure as Example 1, except that 3% (w/v) of choline alfoscerate was further mixed.

### Example 3

An injection composition was prepared by performing the same procedure as Example 2, except that 4% (v/v) of carbonated water was further mixed.

### Example 4

An injection composition was prepared by performing the same procedure as Example 3, except that4% (v/v) of vitamin B1 (Beecom Hexa Injection) was further mixed.

### Example 5

An injection composition was prepared by performing the same procedure as Example 1, except that 0.001% (w/v) of Asparagopsis taxiformis extract was further mixed.

The Asparagopsis taxiformis extract was prepared by adding the Asparagopsis taxiformis whole plant to 70% ethanol two times the weight of the whole plant and extracting at 40° C. for five hours to obtain the extract. The extract was prepared by filtering under a vacuum with a filter paper, concentrating under a vacuum at 40° C., and then freeze-drying.

### Comparative Example 1

An injection composition was prepared by mixing 0.1% (w/v) aminophylline, 0.02% (w/v) procaine, 0.001% (w/v) epinephrine, 300 IU hyaluronidase, and a solvent as a remainder based on the total amount of the composition.

At this time, only distilled water was used as the solvent.

### Comparative Example 2

An injection composition was prepared by mixing 0.1% (w/v) aminophylline, 0.02% (w/v) procaine, 0.004% (w/v) epinephrine, 800 IU hyaluronidase, and a solvent as a remainder.

At this time, only half saline was used as the solvent.

### (Test Example 1) Facial Fat Decomposition Test

In order to confirm the local fat removal effect of the composition of the present invention, a test was performed with 28 adult women in their 40s and 50s using the injection compositions of Examples 1 to 5 and Comparative Examples 1 and 2.

Specifically, the compositions of Examples 1 to 5 and Comparative Examples 1 and 2 were administered to 28 test subjects (each group of 4 subjects; one injection composition used for each group) in an amount of 1 cc per cheek at 3 points, thus a total amount of 6 cc, twice at one-week intervals (week 1 and week 2), and the degree of reduction in the cheek length before the administration and two weeks after the final administration was calculated, and the results are shown as an average in Table 1 below.

TABLE 1
Results of Test Example 1

| | Degree of reduction on the | Degree of reduction on the |
|---|---|---|
| Classification | right (%) | left (%) |
| Example 1 | 8.5 | 8.3 |
| Example 2 | 9.3 | 9.4 |
| Example 3 | 10.4 | 10.2 |
| Example 4 | 10.4 | 10.3 |
| Example 5 | 8.9 | 8.9 |
| Comparative Example 1 | 2.8 | 3.1 |
| Comparative Example 2 | 8.1 | 8.0 |

As shown in Table 1, according to Examples 1 to 5 of the present invention, it was confirmed that the cheek length of the test subjects was reduced by 8% or more before and after the procedure. On the other hand, the degree of reduction in Comparative Examples 1 and 2 was not as good as that of the Examples. It may be because Comparative Example 1 had a lower content of hyaluronidase, and in Comparative Example 1, despite its higher content of hyaluronidase than that of the Examples, the fat decomposition effect was poor because only half saline was used as a solvent.

Therefore, it was confirmed that the Examples of the present invention efficiently remove local fat accumulated on the face.

### (Test Example 2) Abdominal Fat Decomposition Test

A test was performed with 24 abdominal obesity patients with a body mass index (BMI) of 25 or higher among adult men and women aged 20 years or older. Specifically, the compositions of Examples 1 to 5 and Comparative Examples 1 and 2 were administered to 28 test subjects (each group of 4 subjects; one injection composition used for each group) in an amount of 2 cc and at 4 points at 5-cm intervals between points with reference to the navel on the abdomen between the ribs and the pubic bone, in a total amount of 8 cc and totally three times (week 1, week 2, week 3) at one-week intervals.

Then, the reduction of the abdominal circumference was confirmed. The patient's abdominal circumference was measured before the administration, and the abdominal circumference was measured again one week after the three administrations, and the reduction of the abdominal circumference was calculated, and the average value is shown in Table 2 below.

Abdominal circumference reduction value=[1−(abdominal circumference before treatment−abdominal circumference one week after final administration)/abdominal circumference before administration]×100.

In addition, the skin elasticity improvement effect was also measured, and the skin elasticity of the test subjects was confirmed through skin elasticity measurement using a skin elasticity measuring device (cutometer MPA580, Courage+Khazaka, Germany) under the conditions of temperature 24 to 26° C. and humidity 75%. Specifically, the skin elasticity of the lower abdomen region 4 cm next to the navel before the administration was measured, and the skin elasticity of the same region was measured one week after the final administration. Based on the results obtained by using the cutometer MPA580, the average value of the net elasticity value per group was calculated, and the skin elasticity improvement effect was calculated, and the average value is shown in Table 3 below.

Skin elasticity improvement effect=(net elasticity one week after the final administration−net elasticity before the administration)×100.

TABLE 2
Abdominal circumference reduction
value results of Test Example 2

| Classification | Abdominal circumference reduction value |
|---|---|
| Example 1 | 95.0 ± 0.3% |
| Example 2 | 94.6 ± 0.5% |
| Example 3 | 94.1 ± 0.4% |
| Example 4 | 94.9 ± 0.4% |
| Example 5 | 98.6 ± 0.2% |
| Comparative Example 1 | 96.4 ± 0.3% |
| Comparative Example 2 | 95.0 ± 0.3% |

As shown in Table 2 above, it was confirmed that Examples 1 to 5 had a superior abdominal circumference reduction effect than that of Comparative Example 1. However, it was confirmed that Comparative Example 2 had a similar abdominal circumference reduction effect to Example 1.

TABLE 3
Skin elasticity improvement effect results of Test Example 2

| Classification | Abdominal circumference reduction value |
|---|---|
| Example 1 | 0.1 |
| Example 2 | 3 |
| Example 3 | 3.7 |
| Example 4 | 4.3 |
| Example 5 | 3.4 |
| Comparative Example 1 | −0.2 |
| Comparative Example 2 | −2.8 |

As shown in Table 3, it was confirmed that Examples 1 to 5 had excellent effects not only on fat decomposition but also on maintenance and improvement of skin elasticity. On the other hand, it was confirmed that Comparative Example 2 significantly reduced skin elasticity.

### Test Example 3

The degree of occurrence of side effects after the administration was evaluated with the test subjects of Test Example 3, and the average value is shown in Table 4 below. [Very good (5 points), good (4 points), average (3 points), bad (2 points), very bad (1 point)].

TABLE 4
Results of Test Example 3

| | | | | Skin | Skin |
|---|---|---|---|---|---|
| Classification | Redness | Itchiness | Swelling, | depression | sagging |
| Example 1 | 4.5 | 4 | 4 | 5 | 5 |
| Example 2 | 4.5 | 4.5 | 4 | 5 | 5 |
| Example 3 | 4.5 | 4.5 | 4 | 5 | 5 |
| Example 4 | 4.5 | 4.5 | 4 | 5 | 5 |
| Example 5 | 4.5 | 4.5 | 4.5 | 5 | 5 |
| Comparative | 3 | 3 | 3.5 | 3 | 3 |
| Example 1 | | | | | |
| Comparative | 2.5 | 2 | 2.5 | 2.5 | 2 |
| Example 2 | | | | | |

As shown in Table 4, it was confirmed that all of Examples 1 to 5 of the present invention had good or very good side effects with scores of 4.0 or higher. On the other hand, it was confirmed that Comparative Example 2 exhibited side effects with scores of 2.5 or lower.

Those skilled in the art will understand that the present invention may be implemented in other specific forms without changing its technical idea or essential features. Therefore, it should be understood that the above-described embodiments are exemplary in all respects and not limiting. The scope of the present invention is represented by the scope of the claims described below rather than the above detailed description, and all changes or modifications derived from the meaning and scope of the claims and their equivalent concepts should be interpreted as being included in the scope of the present invention.

1. A contour injection composition, comprising:
a solvent, aminophylline, procaine, epinephrine, and hyaluronidase,
wherein, based on the entire composition, the hyaluronidase is included in 500 to 700 IU units, the aminophylline is included in an amount of 0.05% to 0.15% (w/v), the procaine is included in an amount of 0.01% to 0.03% (w/v), the epinephrine is included in an amount of 0.001% to 0.006% (w/v), and the solvent is included as the remainder,
wherein the solvent is half saline and distilled water.

2. The contour injection composition according to claim 1, further comprising:
2% to 4% (w/v) of choline alfoscerate based on the entire composition.

3. The contour injection composition according to claim 2, further comprising:
3% to 6% (v/v) of carbonated water based on the entire composition.

4. The contour injection composition according to claim 3, further comprising:
1% to 5% (v/v) of vitamin B1 based on the entire composition.

5. The contour injection composition according to claim 1,
wherein the half saline and distilled water are included at a volume ratio of 3:7.

6. The contour injection composition according to claim 1, further comprising:
0.001% to 0.01% (w/v) of Asparagopsis taxiformis extract.
